(19) United States
(12) Patent Application Publication
(10) Pub. No.: US 2004/0110214 A1
(43) Pub. Date: Jun. 10, 2004

(54) Chips for detecting DNA and method for detecting DNA using the same

(76) Inventors: Kyu Won Kim, Daejon-Shi (KR); Hae Sik Yang, Daejon-Shi (KR); Yong Beom Shin, Daejon-Shi (KR); Dae Sik Lee, Daejon-Shi (KR); Se Ho Park, Daejon-Shi (KR); Sung Jin Kim, Daejon-Shi (KR); Tae Hwan Yoon, Gyeonggi-Do (KR); Yun Tae Kim, Daejon-Shi (KR)

Correspondence Address: JACOBSON HOLMAN PLLC, 400 SEVENTH STREET N.W., SUITE 600, WASHINGTON, DC, 20004, US

(21) Appl. No.: 10/677,315

(22) Filed: Oct. 3, 2003

(30) Foreign Application Priority Data
Dec. 10, 2002 (KR) 2002-78444

(51) Int. Cl. C12Q 1/68; C12M 1/34

(52) U.S. Cl. 435/6; 435/287.2

(57) ABSTRACT

The present invention provides a chip for detecting DNA and method for detecting DNA using the same. In the chip according to the present invention, a double strand formed by hybridization of signaling DNA and probe DNA is fixed to an electrode, wherein the signaling DNA and probe DNA are complementary each other and either of the two is complementary to sequence of a target DNA, and the complementarity between the target DNA, and the signaling DNA and the probe DNA is different, and electrochemically active compound is bonded to the signaling DNA. Further, the method for detecting DNA according to the present invention induces competitive reaction for performing hybridization of the probe DNA and the signaling DNA, and detects the target DNA by measuring change in an electrochemical signal of the signaling DNA. The chip for detecting DNA and the method for detecting the DNA according to the present invention have the same degree of sensitivity as a conventional detection method without direct marking on the target DNA, and have an advantage like that there exists no need for cleaning step in an electrochemical measurement step suitable for small-sized DNA detection system.

### BACKGROUND OF THE INVENTION

1. Field of the Invention

The present invention relates to a chip capable of detecting DNA and a method for detecting DNA using the same by measuring changes of electrochemical signals from competitive reaction for hybridization, more specifically, to the chip capable of detecting DNA and the method for detecting DNA using the same, without any separate cleaning steps, by making each complementarity of a target DNA, a probe DNA and a signaling DNA labeled electrochemically active materials thereto different and measuring changes of electrochemical signals from competitive reaction from hybridization.

2. Description of the Prior Art

An effective method for detecting DNA to study DNA sequencing or gene expression has been developed along with human genome project. A general method for the DNA sequencing is to use a radioactive isotope.

However, the use of the radioactive isotope for DNA sequencing has been the subject that should be improved in consideration of precise control, and safety problem related to handling of radioactive material. Furthermore, to solve such safety problem, fluorescence method has been subsequently considered, however, some of the fluorescence materials induced mutation, they also need safety consideration like radioactive marker, and high costs are needed for additional equipment for actual fluorescent detection, thereby a new method for detection is still required. What is essential for the new method for detection includes, propriety to a small detection system, and low cost, in addition to sensitivity or specificity for observing single base mutation, etc.

Among several detection methods, an electrochemical detection method is the one that meets the above requirement. The most important thing of the electrochemical detection method is to distinguish electrochemical signals before and after a single strand of probe DNA becomes made to a double strand on an electrode through hybridization with a target DNA. The scheme for electrochemical detecting DNA is classified into two categories, one using electrochemically active property of DNA itself, and second, a one using electrochemically active material rather than DNA. In the former category, a method for utilizing an increase of electrochemical reaction of guanine among bases of DNA during double strand has been widely, however, it has disadvantages that a performance is lowered due to a nonspecific absorption of DNA and electrochemical measurement is performed one time due to irreversible oxidation process of guanine. In the latter category, a change in degree of interaction between the DNA and an electrochemically active material leads to a change in electrochemical signals before and after the hybridization reaction. The latter is also classified into two, one depends on a change in intensity of electrochemical signals representing the change in degree that intercalator or binder producing electrochemical signals is bonded to a double strand hybridized from a single strand of DNA, and the other measures the change of electrochemical signals resulted from the change in complementarity by hybridization of the probe DNA and the target DNA marked with electrochemically active molecules. In the case of direct marking, it has an advantage that it has superior sensitivity to that doesn't use the direct marking, but has a disadvantage that every target DNA should be electrochemically reacted and refined, thereby it is troublesome. In general, these methods have had disadvantages that signal to noise ratio is low due to a non-specific surface absorption and then subsequent processing step like cleaning should necessarily be performed to overcome the above disadvantages. The cleaning step followed by a step for measuring electrochemical signals after hybridization reaction has been an obstacle to implement the small-sized DNA detecting system such as a Lab-on-a-chip.

Therefore, many researches have been conducted for improving the method for detecting DNA, and documents mentioned below have been published.

C. J. Yu et al., according to the Journal of the American Chemical Society, Vol. 123, 11155-11161, 2001, “Electronic Detection of Single-Base mismatch in DNA with ferrocene-modified Probes”, a method for detecting DNA is described that a monolayer of electrically conductive organic compound is mixed with a non conductive organic compound and a DNA layer on an electrode to form two different signaling DNAs, and then a change in electrochemical signals is measured by reacting the signaling DNAs with a wild type of a target DNA. This method has improved the signal to noise ratio by using double signaling DNAs, however, it is not preferable for the small-sized DNA detecting system due to a complex configuration of the chip for detecting DNA.

According to the J. Lahiri et al., Journal of the American Chemical Society, Vol. 124. 2396-2397, 2002, “Method for detection of Single-Base Mismatches using Biomolecular Beacons”, a method is described like that a partially complementary DNA to a probe DNA is labeled with a fluorescent absorbing material and is hybridized with array of probe DNA labeled with fluorescent emitting material, so that a double strand is formed to reduce the fluorescence, and this is immersed into a target DNA solution fully complementary to the probe DNA and is subject to competitive reaction, thereby a change in fluorescence is detected to measure single base mismatches. This method also has an advantage that it has obstacle in detecting signals due to a non-specific absorption of a target DNA less than a general fluorescence method, however, has a disadvantage that it is not yet preferable for the small-sized DNA detecting system due to requirement of an additional equipment for detecting fluorescence.

According to the WO 0250308, a double strand is formed between a DNA 1 complementary to a target DNA and a DNA 2 shorter than DNA 1 and complementary to the DNA 1, and a target DNA is added to and reaction is performed, thereby partially hybridized DNA is separated from the double strand and a stronger double strand is formed between the target DNA and the DNA 1. Fluorescent Resonance Energy Transition (FRET) is applied to the double strand of DNA 1 and DNA 2, and fluorescence is significantly increased when the DNA 2 is separated, thereby the target DNA can be detected. This method can implement detection of single base polymorphism without any complex and stringent steps, however, is not preferable for the small-sized DNA detecting system due to a complex equipment.

As describe above, recent methods are not yet suitable for the small-sized DNA detecting system or for simplified chip for detecting DNA.

Therefore, inventors of the present invention who have conducted researches for small-sized DNA detecting system and a simplified chip for detecting DNA have implemented the following method. In this method for electrochemically detecting DNA, a signaling DNA and a probe DNA are complementary each other and both DNAs are also complementary to base sequence of a target DNA, and the complementarities between the target DNA, the signaling DNA and the probe DNA are different, so that DNA can be detected with high sensitivity without any marking on the target DNA, surface cleaning step for preventing non-specific absorption is not needed, thereby the small-sized DNA detecting system can also be implemented.

### SUMMARY OF THE INVENTION

Therefore, the object of the present invention is to provide DNA detecting of simple structure and having superior sensitivity without marking on the target DNA.

The other object of the present invention is to provide the method for detecting DNA by using the detecting DNA chip having superior sensitivity without any cleaning step and marking step on the target DNA.

To achieve the above objects, the present invention provides the chip for detecting DNA wherein double strands formed by hybridization of signaling DNA and probe DNA are fixed to electrodes, and the signaling DNA and the probe DNA are complementary each other and either of the DNAs is also complementary to a base sequence of a target DNA, and the complementarities between the target DNA and the signaling DNA and the probe DNA are different, wherein the signaling DNA is leveled with an electrochemically active compound.

In the chip for detecting DNA, the electrode can be an electrode array having more than one electrode.

The chip can be made by using oligomers of PNA or LNA instead of the DNA.

A method for forming the chip for detecting DNA can be classified into two, the first one is performed like that the probe DNA is fixed on the electrode by a method for forming a self-assembled monolayer, and said electrode is immersed to the signaling DNA solution, and hybridization reaction is derived by increasing temperature of the solution up to the melting point and decreasing the temperature so that a double strand is formed.

The second one is performed like that temperature of a solution having same amounts of the probe DNA and the signaling DNA is increased up to the melting point and is gradually cooled down, so that a hybridization is derived and a double strand is formed, and the double strand is fixed to an electrode by a method for forming self-assembled monolayer.

Base sequence complementarity of the signaling DNA and the probe DNA with respect to the target DNA is designed by the following two types, so that the chip for detecting DNA in accordance with the present invention can be made.

The first one is performed like that complementarity between base sequences of the probe DNA and the target DNA is higher than that between base sequences of the probe DNA and the signaling DNA.

The second one is performed like that complementarity between base sequences of the signaling DNA and the target DNA is higher than that between base sequences of the probe DNA and the signaling DNA.

Furthermore, in accordance with the present invention, the electrochemically active material which is covalently bonded to the signaling DNA may be preferably selected from a group consisting of polypyridine or polyphenanthroline complex compound of ferrocene, rucenium, or osmium, viologen, hydroquinone, anthraquinone and pyrrolo quinoline quinone.

In addition, the chip for detecting DNA in accordance with the present invention can be made like that at least two double strands that have been formed by using at least two probe DNAs and signaling DNAs corresponding to those probe DNAs are fixed to electrodes of electrode array having at least two electrodes arranged so that simultaneous multiple DNA detection can be performed.

To achieve the other object of the present invention, the present invention provides the method for detecting DNA, which comprises a step for preparing the chip for detecting DNA, a step for deriving competitive reaction among a probe DNA, signaling DNA, and a target DNA by putting a solution of the target DNA to the chip for detecting DNA, and a step for detecting amount and type of the target DNA by measuring a change in electrochemical signal of the signaling DNA before and after the competitive reaction, and wherein the chip for detecting DNA is prepared like that a double strands formed by hybridization of signaling DNA and probe DNA are fixed to electrodes, and the signaling DNA and the probe DNA are complementary each other and either of the DNAs is also complementary to a base sequence of a target DNA, and the complementarities between the target DNA and the signaling DNA and the probe DNA are different, wherein the signaling DNA is labeled with an electrochemically active compound.

In addition, in the step for deriving competitive reaction, it is preferable to repeat that an electrode of the chip is heated near melting temperature of a double strand formed by the signaling DNA and the probe DNA and cooled down.

The amount of the target DNA is quantified by measuring change in electrochemical signal before and after the competitive reaction of the signaling DNA.

The type of the target DNA is determined by performing competitive reaction between a double strand and an unknown target DNA and measuring electrochemical signal produced from the competitive reaction, after forming the double strand of the probe DNA and the signaling DNA with regard to several types of expected base sequences and fixing each of the double strand to each electrode of an electrode array having at least one electrode.

In the method for detecting DNA in accordance with the present invention, multiple DNA detection is possible by using the chip for detecting DNA that is formed by fixing at least two double strands of DNAs produced from at least two different probe DNAs and signaling DNAs corresponding to the probe DNAs to each electrode of an electrode array having at least two electrodes arranged.

Hereinafter, the present invention will be more specifically explained.

The term “probe DNA” used in the present invention has one of thiol, amine, silane, and biotin as a functional group at one end of the probe DNA, and acts to transfer electrochemical signals to the electrode by keeping a signaling DNA bonded through hybridization reaction to an electrode made from one of gold, platinum, ITO (Indium Tin Oxide), and carbon, or acts to bond a target DNA through hybridization reaction.

In addition, the term “target DNA” used in the present invention means a DNA oligomer or product by the PCR (Polymerization Chain Reaction) method that amplify the DNA extracted from chromosomes that has possibility of having required base sequence that needs to be detected.

The signaling DNA has base sequences that is partially complementary so that the hybridization with the probe DNA can be possible, and is prepared through covalent bond reaction between one of ribose, base, and phosphoric acid of DNA, and one of electrochemically active coordinate metal compound such as polypyridine or pholyphenanthroline complex compound of ferrocene, rucenium or osmium, viologen, hydroquinone, anthraquinone, and pyrrolo quinoline quinone. Such a covalent bond reaction can be proceeded in two different ways, wherein the first one is to bond one of the above-mentioned compounds to the 3′ or 5′ end of the signaling DNA synthesized as oligomer type, or one of above-mentioned compound is bonded at the DNA monomer step and then the oligomer is at the synthesis phase. The second one can introduce the above-mentioned compounds with respect to several monomers constituting the whole DNA. This provides a method for increasing electrochemical signals when the signals are insignificant. As described above, DNA detection having high sensitivity can be implemented by using electrochemically active materials covalent boned as marker.

The chip for detecting DNA in accordance with the present invention is made by fixing a double strand formed by hybridization of the probe DNA and the signaling DNA to an electrode. This can be prepared in two different ways.

The first one is to fix the probe DNA to the electrode by a method for forming a self-assembled monolayer, and then the electrode with the probe DNA is immersed to the signaling DNA solution for a predetermined time, and hybridization reaction is derived by increasing temperature of the solution up to the melting temperature (Tm; about 90° C.) and decreasing it so that a double strand is formed and cleaned by buffer solution. Self-assembled monolayer can be prepared by a method comprising the steps of modifying the electrode surface by immersing the electrode into a probe DNA solution containing one of thiol, amine, silane and biotin for a predetermined time, and then immersing electrode modified surface into a mercaptoalkylalcoholic solution in order to fix the thin film of the mercaptoalkylalcohol to a remaining space except that has taken by DNA. The mercaptoalcohoic solution acts to raise the fallen DNA, displaces the weakly bonded probe DNA, and prevents the electrode surface from non-specific absorption of DNA. A mercaptohexanol is a typical example of mercaptoalkylalcohol. A solvent for the signaling DNA and the probe DNA solution can be changed in accordance with the type of DNA, and preferably includes SSC (Saline Sodium-Citrate), PBS (Phosphate Buffered Saline), HBS (HEPES Buffered Saline), TBS (Tris buffered Saline), etc, but not is limited thereto.

The second one is to increase the temperature of the solution having same amount of probe DNA and signaling DNA up to the melting temperature (e.g 90° C.) and gradually decrease it so that hybridization reaction is occurred and a double strand is formed, and to fix it to the electrode by a method for forming a self-assembled monolayer.

A chip for detecting PNA (peptide nucleic acid) or LNA (locked nucleic acid) can be formed by the same method using oligomer of PNA or LNA instead of DNA.

When containing the above-mentioned chip for detecting DNA is dipped into a solution containing target DNA, the DNAs of double strand on the chip and the target DNA in the solution is performed for hybridization. To facilitate the reaction, increasing the solution near the melting temperature of signaling DNA and the probe DNA double strand and cooling down are repeated. The step for increasing temperature can be performed by the heater mounted next to the electrode. In addition, the solvent of the target DNA solution can be changed in accordance with the type of DNA, and preferably includes SSC (Saline Sodium-Citrate), PBS (Phosphate Buffered Saline), HBS (HEPES Buffered Saline), TBS (Tris buffered Saline), etc, but not is limited thereto.

The competitive reaction can be performed in two ways according to design of the base sequence of the probe DNA and the signaling DNA.

The first design is that the complementarity of the base sequence of the probe DNA to the base sequence of the target DNA is higher than to that of the signaling DNA so that the target DNA forms a hybrid of double strand with the probe DNA instead of the signaling DNA through competitive reaction, thereby the signaling DNA is released from the electrode surface to the solution. The method for designing the complementarity between the probe DNA and the target DNA is to include more bases complementary to target DNA in the sequence of probe DNA than those in the sequence of signaling DNA.

The second design is that the complementarity of the signaling DNA to the base sequence of the target DNA is higher than to that of the probe DNA so that the signaling DNA and the target DNA form a hybrid of double strand through competitive reaction, thereby the probe DNA is only left on the electrode surface. The method for designing the complementarity between the signaling DNA and the target DNA higher than that between the probe DNA and the target DNA is to include more bases complementary to target DNA in the sequence of the signaling DNA than those in the probe DNA. The target DNA with bases that the signaling DNA has more than the probe DNA does.

In the above two competitive reactions, the signaling DNA is released from the electrode, and this only occurs when there exits a DNA having a required base sequence, i.e. the DNA having base sequence of the target DNA in the solution and when at least one base sequence is different from the required base sequences, the competitive reaction decreases, and the degree of the signaling DNA releasing from the electrode surface proportionally decreases.

The amount of the signaling DNA on the chip for detecting DNA is reduced compared to that before the competitive reaction. Therefore, the target DNA can be detected by measuring the amount of the signaling DNA within the double strand fixed on the electrode before and after the competitive reaction. The amount of the signaling DNA is derived by measuring the amount of current in accordance with oxidation or reduction of electrochemically active compound bonded to the signaling DNA. The amount of the current can be measured by general method in the art, preferably be measured by predicting a voltage that is response to the electrochemically active material like ferrocene bonded to the signaling DNA and applying proper voltage to a working electrode from a potentiostat by means of a three-electrode type electrochemical device wherein the electrode of the chip is a working electrode, Ag/AgCl is a reference electrode, and Pt wire is an auxiliary electrode. The amount of the target DNA from the unknown sample can be quantified from correlation between the amount of the target DNA and that of the signaling DNA by drawing a calibration curve.

The electrochemical reaction can be observed without cleaning of electrodes, since the amount of the signaling DNA released after competitive reaction does not affect the measurement of electrochemical signal and the solution has an enough electrolyte concentration enough to perform electrochemical reaction when the competitive reaction is induced, so that the used solution can be used as an electrolyte for electrochemical reaction and From the results in accordance with the presence of cleaning step or not, it can be ensured that the difference of electrochemical signals between the signals with cleaning and without cleaning step is not out of the error level.

In addition, a plurality of the chip for detecting DNA can be formed, wherein the chips can be formed by performing hybridization between different probe DNAs and corresponding signaling DNAs, thereby forming different double strands, and fixing the different double strands to each electrode of the electrode array. The multiple chips for detecting DNA can also be applied to the above-mentioned procedure so that multiple DNA detection is enabled. That is, the multiple chips for detecting DNA are put into the solution with target DNAs corresponding to each of those electrodes so that competitive reactions are occurred, and change of the electrochemical signals to each of those electrodes is measured, the target DNAs can be detected at the same time by using a multi-channel potentiostat

Furthermore, when types of the target DNAs are unknown, the probe DNAs and the signaling DNAs having base sequences corresponding to that of predicted several base sequences of target DNA are synthesized by the above-mentioned method, and double strand of the probe DNAs and target DNAs is fixed to an each electrode of the electrode array and put into the unknown target DNA solution for competitive reaction, so that only the electrochemical signal of the corresponding electrode is significantly reduced compared to other electrodes thereby then the type of the DNA is detected. This method can be applied to discriminate single nucleotide polymorphism.

Furthermore, if an electrochemical signal is measured in case of the same competitive reaction in a solution of DNA that is not complementary to the probe DNA, the chip for detecting DNA in accordance with the present invention can determine whether it can detect a complementary DNA.

Hereinafter, embodiments of the present invention will be explained with reference to the accompanying drawings.

### BRIEF DESCRIPTION OF THE DRAWINGS

FIG. 1A shows a structure of a first chip for detecting DNA for inducing competitive reaction between a target DNA and a signaling DNA according to the present invention.

FIG. 1B is a schematic view for competitive reaction among a probe DNA, a signaling DNA, and a target DNA by putting a target DNA solution to the chip for detecting DNA of FIG. 1A.

FIG. 2A shows a structure of a second chip for detecting DNA for inducing competitive reaction between a target DNA and a probe DNA according to the present invention.

FIG. 2B is a schematic view for competitive reaction among a probe DNA, a signaling DNA, and a target DNA by putting a target DNA solution to the chip for detecting DNA of FIG. 2A.

FIG. 3A is a schematic chemical structure of signaling DNA containing ferrocene used for electrochemical measurement according to the present invention.

FIG. 3B is a chemical structure DNA monomer of wherein uracil is used as a base connecting a ferrocene used for electrochemical measurement according to the present invention.

FIG. 4A is a graph showing a result of detecting a target DNA without cleaning step by using the chip for detecting DNA according to the present invention.

FIG. 4B is a graph showing a result detecting a target DNA with cleaning step by using the chip for detecting DNA according to the present invention.

### DESCRIPTION OF THE PREFERRED EMBODIMENTS

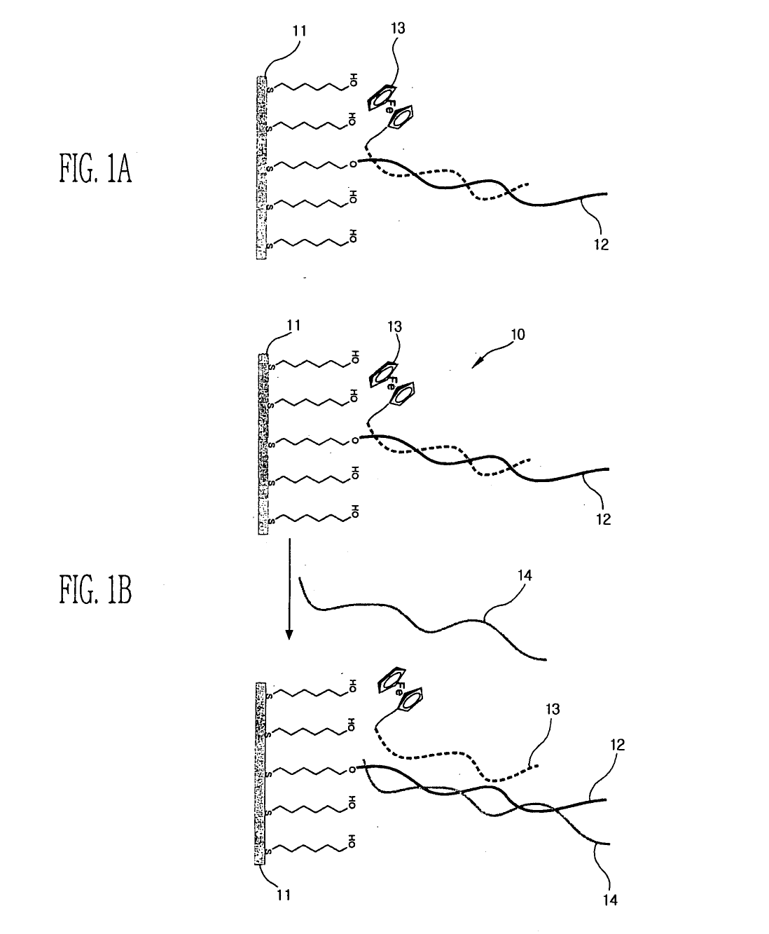

FIG. 1A shows the chips for detecting DNA in which double helix strand of a probe DNA 12 and a signaling DNA 13 is connected to an electrode of electrode array, and FIG. 1B shows the competitive reaction between the target DNA 14 and the signaling DNA 13 when the chips for detecting DNA to be a hybrid with the probe DNA 12 in FIG. 1A is immersed into the target DNA 14 solution. In FIGS. 1A and 1B, the complementarity of the probe DNA 12 with respect to the base sequence of the target DNA 14 is higher than that with respect to the signaling DNA 13.

The chip for detecting DNA in FIG. 1A is prepared as follows. First, gold electrode 11 is immersed in a SSC solution (2 μM) with thiol-drivatized probe DNA for 30 minutes to modify the surface of the electrode 11, and the thin film of mercaptohexanol is fixed on the remaining surfaces of the electrodes by a method for forming a self-assembled monolayer immersing the resulting surfaces to a water solution of 1 mM mercaptohexanol for 30 minutes, and including hybrid reaction by increasing to the melting temperature and then decreasing a temperature after the surfaces are immersed to a SSC solution (10 μM) containing the signaling DNA(13), thereby the double helix strand is made and the SSC is cleaned out.

Alternatively, the chip for detecting DNA of FIG. 1A can be prepared like that temperature of a solution having same amounts of the probe DNA and the signaling DNA is increased up to the melting point and is gradually cooled down, so that a hybridization is derived and a double strand is formed, and the double strand is fixed to an electrode by a method for forming self-assembled monolayer and processed by the method such as a method using mercaptohexanol, as described above.

In FIG. 1B, a solution with a target DNA 14 is put into the chip for detecting DNA 10 in FIG. 1A, and a competitive reaction among the probe DNA 12, signaling DNA 13, and target DNA 14 is performed, and the target DNA 14 is hybridized with the probe DNA 12 instead of the signaling DNA 13, so that the signaling DNA is separated from the electrode surface into the solution.

For an effective process of the competitive reaction, the double strand of the signaling DNA 13 and the probe DNA 12 needs to be heated near to the melting temperature thereof. After heating, each of the three DNA can be separated to a single strand at one time, thereafter the probe DNA 12 and the target DNA 14 form a double strand through hybridization since the complementarity of probe DNA 12 to the target DNA 14 is higher than that to the signaling DNA 13. Thus, the signaling DNA 13 remains in the solution and an electrochemical material on the signaling DNA itself has is separated from the electrode, so that it cannot affect the electrochemical signal of the chip for detecting DNA.

Meanwhile, if the complementarity between the probe DNA and the target DNA that should be detected is lower than that between the signaling DNA and the probe DNA, the signaling DNA and the probe DNA will form a pair through hybridization reaction after competitive reaction, and will be returned to the state before the competitive reaction and the electrical material of signaling DNA will contribute to the electrochemical signal of the chip for detecting DNA. Therefore, the amount of the signaling DNA fixed to the electrode before and after the competitive reaction is measured so that the target DNA is detected.

At this time, the amount of the signaling DNA is obtained by measuring the current of electrochemically active compound bonded to the signaling DNA, wherein the amount of the current is measured by using a three-electrode system type electrochemical device wherein the electrode of the chip for detecting DNA is a working electrode, Ag/AgCl is a reference electrode, and Pt wire is an auxiliary electrode, and a voltage that is response to an electrochemically active material like ferrocene bonded to the signaling DNA is predicted and then a proper voltage therefrom, for example 400-700 mV is applied to the working electrode.

Therefore, the target DNA is detected by a calibration curve from the correlated between the amount of target DNA and the amount of the signaling DNA through measurement of electrochemical signals.

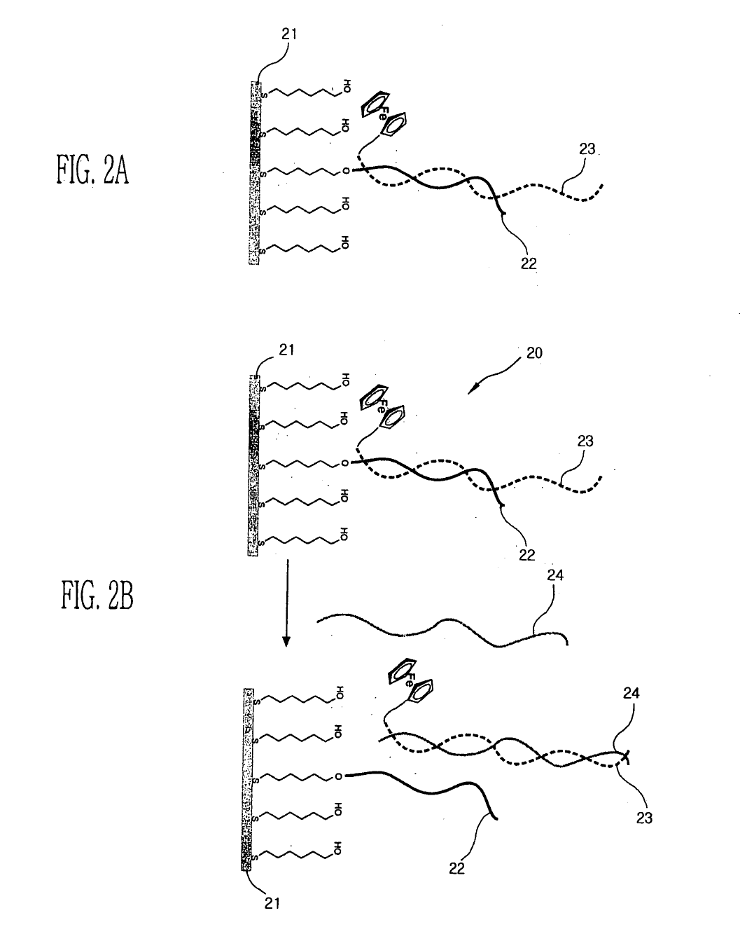

FIGS. 2A and 2B show the case that the complementarity of the base sequence of the signaling DNA 23 to the base sequence of the target DNA 23 is designed as higher than to the probe DNA 22, wherein FIG. 2A shows a structure of the chip for detecting DNA to which the double strand of the probe DNA 22 and the signaling DNA 23 is fixed to the electrode, and FIG. 2B shows the competitive reaction among a probe DNA 22, a signaling DNA 23, and a target DNA 24 by putting a target DNA 24 solution to the chip 20 for detecting DNA of FIG. 2A.

FIG. 2B shows that when the base sequence of the signaling DNA 23 is determined, the complementarity of the base sequence of the signaling DNA 23 to the base sequence of the target DNA 24 is designed as higher than that of the base sequence of the signaling DNA 23 to the base sequence of the probe DNA 22, and the signaling DNA 23 is subject to hybridization to form a double strand with the target DNA 24 instead of the probe DNA 22 after competitive reaction to form a double strand, so that the double strand of the target DNA 24 and the signaling DNA 23 is separated from the electrode surface into the solution.

This case can also be applied for DNA detection by using the same exemplary method as described above with regard to FIGS. 1A and 1B.

In the preferred embodiments of FIG. 1 and FIG. 2, ferrocene, one of metal coordination compounds, is used as electrochemically active material of signaling DNA (13,23)

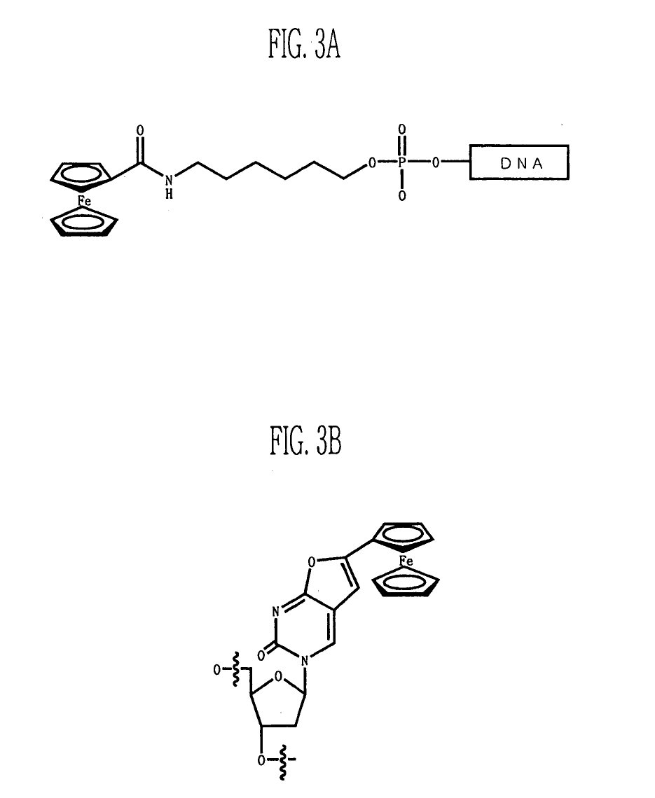

FIG. 3A examplifies a signaling DNA made by a method synthesizing oligomer of DNA in DNA synthesizer, then, introducing a primary amine at the end followed by covalently bonding ferrocene carboxylate through amide bonding.

FIG. 3B shows an example resulted from covalent bonding and crystallization reaction of Uracil, one of DNA monomer, and ferrocene.

These kinds of monomers can be introduced into the base sequence at the desired position or bonded at the end of DNA in DNA synthesizer as phosphoramidite form.

When the chip for detecting DNA shown in FIG. 1A is configured by using the signaling DNA shown in FIG. 3A, the ferrocene is oxidized near 650 mV with respect to the Ag/AgCl reference electrode, and when the DNA is synthesized by using the monomer shown in FIG. 3B, the ferrocene is oxidized near 600 mV.

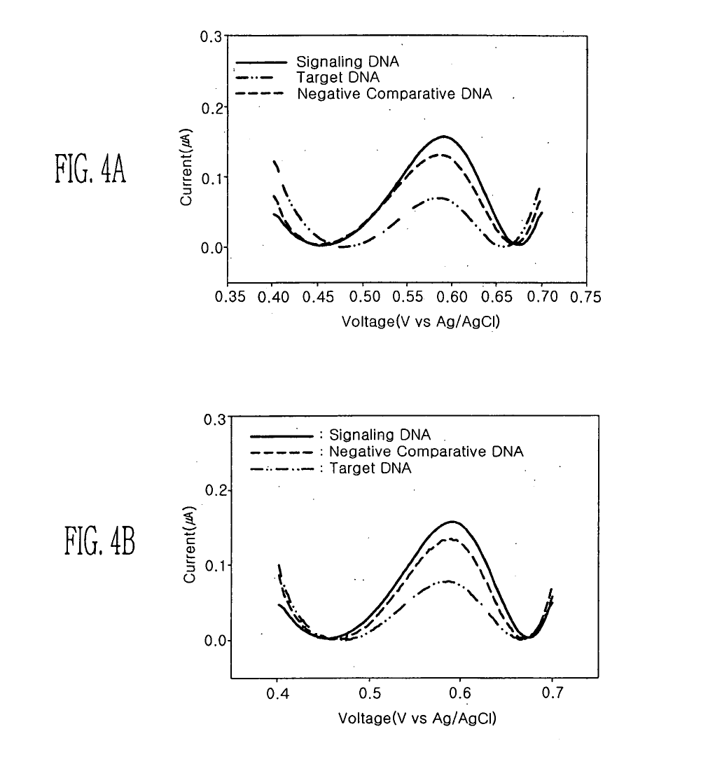

FIG. 4 shows a graph as a result of a target DNA detection by using the chip for detecting DNA according to the present invention. Experiment method is as follows.

Experiment Method

The signaling DNA, target DNA, negative comparative DNA, and probe DNA having the base sequences shown in table 1 were prepared. In the signaling DNA, a monomer of FIG. 3B having ferrocene was bonded to a 5′ end of the signaling DNA. And then, the chip for detecting DNA shown in FIG. 1A was made by using the signaling DNA and the probe DNA. Competitive reaction was performed by putting the target DNA solution and negative comparative DNA solution into the detecting DNA. Before hybridization through competitive reaction, amount of oxidation current of the ferrocene of the signaling DNA was measured by using three-electrode system type electrochemical device wherein the electrode of the chip is a working electrode, Ag/AgCl is a reference electrode, and Pt wire is an auxiliary electrode, and applying 400-700 mV voltage to the working electrode. After competitive reaction between the target DNA and the negative comparative DNA, amount of current of the signaling DNA with respect to each of the target DNA and the negative comparative DNA was measured using the same device as above by applying 300-700 mV voltage to the working electrode, for the case that the cleaning step was performed or not. FIG. 4A shows a graph of baseline-corrected differential pulse voltammogram (DPV) as a result when a target DNA is detected without cleaning step and FIG. 4B shows a result graph when a target DNA is detected after cleaning step.

In FIG. 4A, the solid line represents an amount of current of the signaling DNA hybridized with the probe DNA in the chip for detecting DNA before competitive reaction, the dotted line represents an amount of current of the signaling DNA after competitive reaction with the negative comparative DNA, and the double dotted line represents an amount of current of the signaling DNA after competitive reaction with the target DNA. FIG. 4B also has corresponding lines. As can be expected, the amount of current for the target DNA has been drastically reduced, and FIGS. 4A and 4B show very similar graphs, so that it becomes sure that very similar electrochemical signals are obtained in regardless of cleaning step.

Meanwhile, by performing competitive reaction between the chip for detecting DNA according to the present invention and the target DNA with different concentration and measuring electrochemical signals therefrom and drawing calibration curve for the change of the concentration, unknown amount of the target DNA can be electrochemically quantified.

TABLE 1

| | |
|---|---|
| DNA type | Base sequence |

| | |
|---|---|
| Signaling DNA | 5′ FeFe G CGG GGA GCA G 3′ |
| Target DNA | 5′ GCC ACG CGG GGA GCA G 3′ |
| Negative comparative DNA | 5′ CCG ATG GAC GCA CCG G 3′ |
| Probe DNA | 5′ CTG CTC CCC GCG TGG C 3′ |

Although the present invention has been described in conjunction with the preferred embodiment, the present invention is not limited to the embodiments, and it will be apparent to those skilled in the art that the present invention can be modified in variation within the scope and the spirit of the invention.

According to the chip for detecting DNA and the method for detecting DNA using the same of the present invention, a disadvantage such that a cleaning step is required before electrochemical measurement due to an interference of signal caused by non-specific adsorption through direct narking of target DNA of electrochemical detecting. High cost is required due to many equipments when the DNA is detected by fluorescence and that small-sized DNA detecting system can not be introduced are overcome. In the present invention, each target DNA needs not to be marked, and an electrochemically active material is bonded to the signaling DNA as marker, thereby DNA detection having high sensitivity can be implemented. In addition, the cleaning step to remove non-specific adsorption in the conventional electrochemical detection method is not required any more, so that the small-sized DNA detecting system can be implemented, and thereby fluid control can be minimized during DNA detection when it is applied to a DNA detecting system such as a Lab-on-a-chip.

What is claimed is:

1. A chip for detecting DNA,
wherein a double strands formed by hybridization of signaling DNA and probe DNA is fixed to electrodes, and the signaling DNA and the probe DNA are complementary each other and either of the DNAs is also complementary to a base sequence of a target DNA, and the complementarities between the target DNA and the signaling DNA and the probe DNA are different, wherein the signaling DNA is labeled with an electrochemically active compound.

2. The chip for detecting DNA as claimed in claim 1,
wherein said electrode of the chip for detecting DNA is an array form having at least one electrode.

3. The chip for detecting DNA as claimed in claim 1,
wherein the chip for detecting DNA is made by fixing the probe DNA on the electrode by a method for forming a self-assembled monolayer, immersing said electrode to the signaling DNA solution, and deriving hybridization reaction by increasing temperature of the solution up to the melting point and decreasing it so that a double strand is formed.

4. The chip for detecting DNA as claimed in claim 1,
wherein the chip for detecting DNA is made by drawing hybridization reaction by increasing temperature of a solution containing same amounts of the probe DNA and the signaling DNA up to the melting point and cooling down gradually, so that a double strand is formed, and fixing the double strand on an electrode by a method for forming self-assembed monolayer.

5. The chip for detecting DNA as claimed in claim 1,
wherein complementarity between base sequences of the probe DNA and the target DNA is higher than that between base sequences of the probe DNA and the signaling DNA.

6. The chip for detecting DNA as claimed in claim 1,
wherein complementarity between base sequences of the signaling DNA and the target DNA is higher than that between base sequences of the probe DNA and the signaling DNA.

7. The chip for detecting DNA as claimed in claim 1,
wherein the electrically active material that is bonded to the signaling DNA is at least one selected from a group consisting of polypyridine or polyphenanthroline complex compound of ferrocene, rucenium, or osmium, viologen, hydroquinone, anthraquinone, and pyrrolo quinoline quinone.

8. The chip for detecting DNA as claimed in claim 1,
wherein the chip for detecting DNA is prepared by fixing at least two double strands that have been formed by using at least two probe DNAs and signaling DNAs corresponding to those probe DNAs to electrodes of electrode array having at least two electrodes arranged so that simultaneous multiple DNA detection can be performed.

9. A method for detecting DNA, comprising:
preparing a chip for detecting DNA, wherein a double strands formed by hybridization of signaling DNA and probe DNA is fixed to electrodes, and the signaling DNA and the probe DNA are complementary each other and either of the DNAs is also complementary to a base sequence of a target DNA, and the complementarities between the target DNA and the signaling DNA and the probe DNA are different, wherein the signaling DNA is labeled with an electrochemically active compound;
deriving competitive reaction among the probe DNA, the signaling DNA, and the target DNA by putting a solution of the target DNA to the chip for detecting DNA; and
detecting amount and type of the target DNA by measuring a change of the electrochemical signal of the signaling DNA before and after the competitive reaction.

10. The method for detecting DNA as claimed in claim 9,
in said step for deriving competitive reaction, heating electrode of the chip near to the melting temperature of a double strand formed by the signaling DNA and the probe DNA and cooling is repeated.

11. The method for detecting DNA as claimed in claim 9,
wherein the amount of the target DNA is quantified by measuring change of electrochemical signal before and after the competitive reaction of the signaling DNA.

12. The method for detecting DNA as claimed in claim 9,
wherein the type of the target DNA is determined by performing competitive reaction between a double strand and an unknown target DNA and measuring electrochemical signal produced from the competitive reaction, on a chip for DNA detection having electrode array of at least one electrode formed by fixing several double strands of the probe DNAs and the signaling DNAs corresponding to several types of expected base sequence of target DNAs.

13. The method for detecting DNA as claimed in claim 9,
wherein multiple DNA detection is performed by using the chip for detecting DNA that is formed by fixing at least two double strands of DNAs produced from at least two different probe DNAs and signaling DNAs corresponding to the probe DNAs to each electrode of an electrode array having at least two electrodes arranged.
